(19) United States
(12) Patent Application Publication
(10) Pub. No.: US 2020/0222469 A1
(43) Pub. Date: Jul. 16, 2020

(54) COMPOSITIONS FOR WOUND TREATMENT

(71) Applicant: BRIGHTWAKE LIMITED, Nottingham (GB)

(72) Inventor: Lewis COTTON, Papplewick, Nottingham (GB)

(21) Appl. No.: 16/650,623

(22) PCT Filed: Sep. 26, 2018
(86) PCT No.: PCT/GB2018/052737

§ 371 Date: Mar. 25, 2020

(30) Foreign Application Priority Data
Sep. 27, 2017 (GB) 1715639.9

(51) Int. Cl. A61K 35/644; A61L 26/00; A61L 15/26

(52) U.S. Cl. CPC A61K 35/644; A61L 26/0057; A61L 26/0066; A61L 2300/404; A61L 26/0095; A61L 15/26; A61L 2300/30; A61L 26/008

(57) ABSTRACT

A wound treatment composition comprises honey and a gelling agent dispersed in water, and forms a gel on contact with wound exudate. The composition may be prepared by dispersing the gelling agent in water and mixing the water and gelling agent mixture with honey. A kit comprises the wound treatment composition and a secondary dressing.

The present invention relates to fluid compositions for the treatment of wounds, and in particular to compositions based on honey.

Liquid honey is generally a supersaturated solution of sugars in water, the water content of the honey typically being 15-20% w/w. Honey has long been known to have healing properties. In particular, certain honeys such as manuka honey, produced by bees from the nectar of the plant species Leptospermum scoparium, have been shown to have high antibacterial, non-peroxide activity which inhibits the growth of various species of bacteria. As such, honey can support wound healing through its anti-inflammatory action, its natural antibacterial properties, its debriding action and its stimulatory effect on granulation and epithelialisation. In fact, honey has been shown to have considerable wound- and ulcer-healing capacity and strong antimicrobial capacity even in moist healing environments.

Wound dressings comprising honey are known in the art. However, the honey capacity of the currently available dressings is limited and often lower than would be desirable. One reason for this is the fluid nature of honey, which results in the honey being liable to flow away from the site of the wound. In addition, if a wound produces a significant volume of exudate, the exudate may dilute the honey and exacerbate the problems associated with its fluidity. In addition, wound dressings have fixed dimensions, which may not match the size and shape of the wound to which the dressing is applied. Also, wounds may occur on irregularly-shaped parts of the body to which it is difficult to securely affix a wound dressing.

A further problem associated with the use of honey in wound dressings is that, particularly where the wound-contacting part of the dressing comprises a gelling material, gel-blocking can occur. This is where a continuous layer of gel is formed at the dressing/wound interface and prevents the transfer of honey to the wound.

An alternative to the traditional structured wound dressing which provides more flexibility for application is the use of a gel wound treatment composition. Gels have the advantage of keeping the wound moist, which facilitates wound healing. However, a moist wound environment can also encourage the growth of bacteria, and if the surrounding skin is not kept dry then maceration of the healthy skin can occur.

A honey-containing wound treatment composition is described in WO2007/048193. This composition comprises honey and a suspended particulate gelling agent. Such compositions may be applied to wounds and, on contact with wound exudate, the particles of gelling agent swell and transform into a gel.

Honey-containing wound treatment gels are also commercially available, for example Manuka Honey Wound Gel (Manuka Health New Zealand, www.honeywoundcare.com) and MediHoney® Antibacterial Wound Gel. Manuka Honey Wound Gel comprises manuka honey combined with gelling agents to provide a thicker consistency, and MediHoney® is a combination of medical grade honey with natural waxes and oils. These products contain no added water, and due to their thick consistency can be difficult to apply effectively and evenly to the wound.

There has now been devised an improved composition for treating wounds which overcomes or substantially mitigates the above-mentioned and/or other shortcomings or disadvantages associated with the prior art.

According to the invention there is provided a wound treatment composition comprising honey and a gelling agent, the honey and gelling agent being dispersed in water, wherein the wound treatment composition forms a gel on contact with wound exudate.

The composition of the invention is particularly advantageous as, prior to application to the wound, it is in a flowable form enabling effective application to the whole wound area. Once applied, contact with wound exudate causes the composition to become gelatinous. This forms a barrier between the wound and the atmosphere, protecting it from contamination. This barrier provides a moist wound environment, which is known to be important for the healing process, while the antibacterial components of the honey used to produce the composition prevent the growth of bacteria.

The term “dispersed” is intended, in the context of the invention, to mean that the honey and gelling agent are mixed with the water in such a way as to produce a homogeneous composition. Most commonly, the honey and gelling agent will be dissolved in the water, so that the composition of the invention is a solution of honey and gelling agent.

The water used to prepare the composition may account for between about 5% and about 75% w/w of the composition, or between about 10% and 60% w/w of the composition, more preferably between about 10% and about 50% w/w of the composition. Most preferably, the water used to prepare the composition accounts for between about 15% and about 50% w/w of the composition. Thus, water used to prepare the composition may account for about 5%, 6%, 7%, 8%, 9%, 10%, 11%, 12%, 13%, 14%, 15%, 16%, 17%, 18%, 19%, 20%, 21% 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, or about 75% w/w of the composition. Typically, the water used to prepare the composition may account for from about 5%, 10% or 15% w/w of the composition, to about 50%, 60% or 70% w/w of the composition.

Thus; the total water content of the composition (taking into account the water content of the honey) may be between about 9% and about 80% w/w, or between about 14% and about 67% w/w, or between about 14% and about 59% w/w, more preferably between about 17% and about 59% w/w. Thus, the total water content of the composition may be 9%, 10%, 11%, 12%, 13%, 14%, 15%, 16%, 17%, 18%, 19%, 20%, 21% 22%, 23%, 24%, 25%, 26%, 27%, 28%, 29%, 30%, 31%, 32%, 33%, 34%, 35%, 36%, 37%, 38%, 39%, 40%, 41% 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73% 74%, 75%, 76%, 77%, 78%, 79% or about 80% w/w. Typically, the total water content of the composition is from about 9%, 14% or 17% w/w, to about 59%, 67% or 80% w/w.

The water used to prepare the composition of the invention is preferably deionised water.

Honey has long been known to be effective in treating wounds, with records of such use dating from at least 2000 years ago. More recently, research has shown it to have potent antimicrobial, antifungal and anti-inflammatory properties, and to be able to stimulate lymphocytic and phagocytic activity within the body. Further, honey has been demonstrated to assist in the debridement of necrotic tissue, and to stimulate the growth of new tissue. In terms of its antibacterial activity, honey has been reported to have an antibacterial effect on more than 60 species of bacteria, including aerobes, anaerobes, Gram-positive and Gram-negative bacteria. In particular, honey has been shown to be effective against antibiotic-resistant strains of bacteria including MRSA.

Without wishing to be bound by theory, it is believed that the antibacterial activity of honey is partly due to the release of low levels of hydrogen peroxide, a well-known antimicrobial agent. As the production of hydrogen peroxide is stimulated by dilution of the honey (eg by wound exudate), honey has the distinctive property of becoming more active on dilution, rather than less.

Many studies have shown that the maintenance of a moist wound environment aids in wound healing. However, a moist environment also promotes the growth of bacteria, and the prevention of infection is therefore a serious concern. The addition of honey to a wound treatment composition thus inhibits bacterial growth within the moist environment.

Honey is produced worldwide from many different floral sources, and its antibacterial activity varies with the source of the honey and the processing it has undergone. For example, lotus honey in India is reputed to be good for eye diseases, whereas manuka honey, a monofloral honey produced by bees from the nectar of the manuka bush, is known for its antiseptic properties. The manuka plant is part of the genus Leptospermum, and honeys produced from the nectar of plants of this genus, such as manuka or jelly bush honey, are known for their particularly strong anti-bacterial properties. Preferably, the honey used in the present invention is produced from the nectar of plants of the genus Leptospermum. More preferably, the honey is manuka honey or jelly bush honey.

The honey used to prepare the composition may account for between about 25% and about 90% w/w of the composition, or between about 30% and 80% w/w of the composition, more preferably between about 40% and about 80% w/w of the composition. Most preferably, the honey used to prepare the composition accounts for between about 60% and about 70% w/w of the composition. Thus, the honey may account for about 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81% 82%, 83%, 84%, 85%, 86%, 87%, 88%, 89% or about 90% w/w of the composition.

Suitable gelling agents are known in the art, and include alginates, pectin, chitosan, hydroxyethylcellulose, carbomers, poloxamers or chemically-modified cellulose (CMC). Preferably the gelling agent is an alginate or pectin. More preferably, the gelling agent is an alginate, most preferably sodium alginate.

Alginates are high molecular weight, hydrophilic polymers, which are derived from seaweed and which form a gel on contact with aqueous fluids. Their hydrophilic nature encourages the absorption of liquid such as wound exudate, making them extremely useful in wound treatment.

The alginate polymer is formed of two basic monomeric units, mannuronic acid and guluronic acid. Differing proportions of these units in the polymer alter the properties of the alginate. In addition to this, alginate polymers are associated with cations, and are normally produced in the form of sodium alginate, calcium alginate or a sodium/calcium alginate mix. Other forms, such as potassium alginate, are also known. Sodium alginate is water-soluble, and is thus particularly preferred for use in the present invention.

Pectins are a family of complex polysaccharides comprising 1,4-linked γ-D-galactosyluronic residues, found primarily in the cell walls of terrestrial plants. Pectins can be separated into two main groups, which have different gelling properties: low-methoxy and high-methoxy pectins. Low-methoxy pectins are pectins in which less than half the carbonyl groups in the chain of galacturonic residues are esterified with methanol. Low-methoxy pectins can form a gel in the presence of divalent cations (eg calcium), due to non-covalent ionic interactions between blocks of galacturonic acid residues and the divalent ion. High-methoxy pectins are those in which more than half of the carbonyl groups have been esterified with methanol. Such pectins can gel in the presence of sugar and acid, forming two-dimensional networks of pectin molecules in which the solvent (water) is immobilised with the sugar and acid co-solutes.

Preferably, the gelling agent is present at a concentration of at least 0.5% w/w, more preferably at least about 1% w/w, more preferably at least about 2.5% w/w, at least about 5% w/w, at least about 7.5% w/w, or at least about 10% w/w. Preferably, the gelling agent is present at a maximum concentration of 30% w/w, or 20% w/w, or 15% w/w. For example, the gelling agent may be present at a concentration between 0.5% and 30% w/w, or at between 1% and 20% w/w, or between 1% and 15% w/w, or between 5% and 15% w/w, or between 10% and 15% w/w.

Additional agents known to enhance or assist wound healing may also be included in the composition. For example, the composition may also comprise one or more additional antimicrobial agents, antiseptic agents, antifungal agents, anti-inflammatory agents, vitamins and/or minerals.

One additional antimicrobial that may be incorporated into the wound treatment composition of to the invention is polyhexamethylene biguanide (PHMB; also known as polyaminopropyl biguanide). PHMB is available as a 20% aqueous solution under the trade name COSMOCIL® CQ from Arch Personal Care Products, 70 Tyler Place, South Plainfield, N.J. 07080, USA.

Another additional antimicrobial that may be incorporated is manuka oil. Manuka oil is produced from the manuka plant. Manuka oil has been found to have antibacterial, anti-fungal and anti-inflammatory properties, and has been used to combat skin irritation and infections. It is effective against a range of bacteria, including Gram-positive and Gram-negative bacteria, and antibiotic resistant strains of bacteria including MRSA. Preferably the manuka oil is fractionated manuka oil. By fractionating the manuka oil the fractions with particularly high antibacterial, antifungal and anti-inflammatory properties may be isolated. It is then possible to include a smaller quantity of manuka oil in the composition whilst retaining the desirable properties.

Other antimicrobial agents known in the art may also be incorporated into the composition of the invention.

Vitamins and minerals that may be included in the wound treatment composition include vitamin A, vitamin C, vitamin E and zinc.

If included, the additional agent or agents may be present in the wound treatment composition at a concentration of at least 0.1% w/w, more preferably at least about 0.5% w/w, more preferably at least about 1% w/w. Preferably, the additional agent or agents are present at a maximum concentration of 15% w/w, or 10% w/w, more preferably the additional agent or agents are present at a maximum concentration of 9% w/w, 8% w/w, 7% w/w, 6% w/w, 5% w/w, 4% w/w, 3.5% w/w, or 3% w/w. For example, the additional agent or agents may be present at a concentration of between about 0.1% and 15% w/w, or between about 0.5% and 10% w/w, or between about 0.5% and 9% w/w, or between about 0.5% and 8% w/w, or between about 1% and 7% w/w, or between about 1% and 6% w/w, or between about 1% and 5% w/w, or between about 1% and 4% w/w.

The wound treatment composition may comprise one or more additional antimicrobial agents, eg manuka oil and/or PHMB.

In use, the wound treatment composition contacts wound exudate which leads to the composition becoming gelatinous. Without wishing to be bound by theory, it is believed that cations present in wound exudate allow the gelling agent to cross link and thus thicken the composition. If insufficient wound exudate is present, or promotion of an external barrier is required, a suitable cation-containing solution may also be applied to the wound treatment composition. If used, such a solution is preferably sprayed onto the wound treatment composition. Any such solution is preferably sterile.

In general, the wound treatment composition of the invention is flowable, such that it can readily be dispensed onto, and spread over, a wound. That said, the viscosity of the composition may vary within quite wide limits. In some embodiments, the composition is sufficiently stiff to substantially retain its shape when dispensed onto a wound, yet not so stiff that it cannot be readily spread over the wound by the application of gentle manual force. In other embodiments, the composition is less viscous, such that it spreads spontaneously upon dispensing.

As a general rule, compositions that contain higher proportions of water and/or lower proportions of gelling agent will have lower viscosity, and will spread spontaneously upon application of low shear force. Compositions containing less water and/or more gelling agent will be of greater viscosity, and will require somewhat greater shear force to spread them over the wound area.

As noted above, the total water content of the composition of the invention (ie the amount of water in which the honey and gelling agent are dispersed plus the water present in the honey itself) may vary within wide limits, as may the quantity of gelling agent present.

Typically, the total water content of the composition may be about 15% to 60% w/w, and the amount of gelling agent about 2% to 20% w/w, with the balance of the composition being made up of the other components of the honey (ie the components of honey apart from the water present in the honey) and any other additives, such as one or more additional antimicrobial agents.

In one group of embodiments, of relatively high viscosity, the composition comprises from about 20% to 40% w/w water, from about 10% to 20% w/w gelling agent, and up to 2% w/w additional antimicrobial agent, the balance of the composition being made up substantially or completely of the components of the honey other than water. The additional antimicrobial agent is preferably manuka oil and the honey used is preferably manuka honey.

In another group of embodiments, of relatively low viscosity, the composition comprises from about 40% to 60% w/w water, from about 3% to 10% w/w gelling agent, and up to 3% w/w additional antimicrobial agent, the balance of the composition being made up substantially or completely of the components of the honey other than water. Again, the additional antimicrobial agent is preferably manuka oil and the honey used is preferably manuka honey.

The composition of the invention may be sterilised in order to reduce the risk of contaminants, eg bacteria, entering the wound environment. Sterilisation methods are known in the art, and any suitable sterilisation method may be used. Such methods may include steam sterilisation, heat sterilisation and/or radiation sterilisation. Preferably, the composition of the invention is sterilised using steam sterilisation, most preferably in a steam baffled system.

The composition may be supplied in a form that is ready to apply directly to a wound, and is packaged in a tube or other suitable container. When it is applied directly to a wound, a secondary dressing may be applied over the top.

Thus, in a second embodiment of the invention, there is provided a kit comprising a wound treatment composition and a secondary dressing, said wound treatment composition comprising honey and a gelling agent, the honey and gelling agent being dispersed in water, wherein the wound treatment composition forms a gel on contact with wound exudate.

The secondary dressing may be any suitable dressing known in the art. For example, the secondary dressing may be, or may comprise, an air- and moisture vapour-permeable plastics film, preferably a polyurethane film.

The wound treatment composition may be manufactured by any suitable method. For instance, the gelling agent may first be mixed with water, followed by mixing of the honey and the gelling agent/water mixture. The honey may be added to the gelling agent/water or vice versa. Mixing of the honey and gelling agent/water is typically carried out under high shear mixing conditions. To facilitate mixing, the honey may be warmed, typically to a temperature of between 30° C. and 50° C., eg about 40° C. Any additional agents may be added to the composition at any suitable stage of the manufacturing process.

The invention will now be illustrated, by way of example only, with reference to the following Examples.

### EXAMPLE 1

| Ingredient | Mass | | % w/w |
|---|---|---|---|
| Deionised water | 15 | kg | 19.74 |
| Honey | 50 | kg | 65.79 |
| Sodium Alginate | 10 | kg | 13.16 |
| Manuka Oil | 1 | kg | 1.31 |
| TOTAL | 76 | kg | 100% |

### Manufacturing Method:

1. Alginate powder was slowly added to deionised water and mixed at 6000 rpm for 20 minutes using a Silverson Shear Mixer.
2. Manuka oil was added to the alginate/water mixture.
3. Honey was heated to 40° C.
4. The alginate/water/manuka oil mixture from Step 2 was slowly added to the honey from Step 3, and mixed at 6000 rpm for 20 minutes using a Silverson Shear Mixer.

### EXAMPLE 2

| Ingredient | Mass | | % w/w |
|---|---|---|---|
| Deionised water | 23.91 | kg | 47.15 |
| Honey | 23.91 | kg | 47.15 |
| Sodium Alginate | 1.89 | kg | 3.73 |
| Manuka Oil | 1 | kg | 1.97 |
| TOTAL | 50.71 | kg | 100% |

### Manufacturing Method:

1. Alginate powder was slowly added to deionised water and mixed at 6000 rpm for 20 minutes using a Silverson Shear Mixer.
2. Honey was heated to 40° C.
3. Manuka oil was slowly added to the honey, and mixed at 6000 rpm for 15 minutes using a Silverson Shear Mixer.
4. The alginate/water mixture from Step 1 was slowly added to the honey/manuka oil mixture from Step 3, and mixed at 6000 rpm for 20 minutes using a Silverson Shear Mixer.

1. A wound treatment composition comprising honey and a gelling agent, the honey and gelling agent being dispersed in water, wherein the wound treatment composition forms a gel on contact with wound exudate.

2. A wound treatment composition according to claim 1, wherein the water used to prepare the composition accounts for between about 5% and about 75% w/w of the composition.

3. A wound treatment composition according to claim 2, wherein the water used to prepare the composition accounts for between about 10% and about 50% w/w of the composition.

4. A wound treatment composition according to claim 3, wherein the water used to prepare the composition accounts for about 15% and about 25% w/w of the composition.

5. A wound treatment composition according to claim 2, wherein the honey is produced from nectar of plants of the genus Leptospermum.

6. A wound treatment composition according to claim 5, wherein the honey is manuka honey or jelly bush honey.

7. A wound treatment composition according to claim 6, wherein the honey is manuka honey.

8. A wound treatment composition according to claim 1, wherein the honey used to prepare the composition accounts for between about 25% and about 90% w/w of the composition.

9. A wound treatment composition according to claim 8, wherein the honey used to prepare the composition accounts for between about 40% and about 80% w/w of the composition.

10. A wound treatment composition according to claim 9, wherein the honey used to prepare the composition accounts for between about 60% and about 70% w/w of the composition.

11. A wound treatment composition according to claim 1, wherein the gelling agent is an alginate or pectin.

12. A wound treatment composition according to claim 11, wherein the gelling agent is an alginate.

13. A wound treatment composition according to claim 12, wherein the gelling agent is sodium alginate.

14. A wound treatment composition according to claim 1, wherein the gelling agent is present at a concentration of at least 0.5% w/w.

15. A wound treatment composition according to claim 14, wherein the gelling agent is present at a concentration of at least about 5% w/w.

16. A wound treatment composition according to claim 15, wherein the gelling agent is present at a concentration of at least about 10% w/w.

17. A wound treatment composition according to claim 14, wherein the gelling agent is present at a maximum concentration of 30% w/w.

18. A wound treatment composition according to claim 1, which further comprises one or more additional agents selected from antimicrobial agents, antiseptic agents, antifungal agents, anti-inflammatory agents, vitamins and/or minerals.

19. A wound treatment composition according to claim 18, wherein the one or more additional agents comprise PHMB and/or manuka oil.

20. A wound treatment composition according to claim 19, wherein the one or more additional agents comprise manuka oil.

21. A wound treatment composition according to claim 18, wherein the one or more additional agents are present at a concentration of at least about 0.5% w/w.

22. A wound treatment composition according to claim 18, wherein the one or more additional agents are present at a maximum concentration of 15% w/w.

23. A wound treatment composition according to claim 1, which comprises from about 20% to 40% w/w water, from about 10% to 20% w/w gelling agent, and up to 2% w/w additional antimicrobial agent, the balance of the composition being made up substantially or completely of the components of the honey other than water.

24. A wound treatment composition according to claim 1, which comprises from about 40% to 60% w/w water, from about 3% to 10% w/w gelling agent, and up to 3% w/w additional antimicrobial agent, the balance of the composition being made up substantially or completely of the components of the honey other than water.

25. A wound treatment composition according to claim 23, wherein the additional antimicrobial agent is manuka oil and the honey is manuka honey.

26. A kit comprising a wound treatment composition and a secondary dressing, said wound treatment composition comprising honey and a gelling agent, the honey and gelling agent being dispersed in water, wherein the wound treatment composition forms a gel on contact with wound exudate.

27. A kit according to claim 26, wherein the secondary dressing is an air- and moisture vapour-permeable plastics film.

28. A kit according to claim 27, wherein the air- and moisture vapour-permeable plastics film is a polyurethane film.

29. A method for producing the wound treatment composition of claim 1, comprising the steps of:
a) dispersing the gelling agent in water; and
b) mixing the water/gelling agent mixture with the honey.

30. A method according to claim 29, wherein in step b) the water/gelling agent mixture is added to the honey.

31. A method according to claim 29, wherein the honey is warmed prior to step b).

32. A method according to claim 29, wherein an additional antimicrobial agent is added to the water and/or to the honey prior to step b).
